SEQUENCE LISTING (26 sequences)

SEQ ID NO: 1 - RNA, 386 nt, Hepatitis C virus
gccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacacc

SEQ ID NO: 2 - RNA, 804 nt, Artificial sequence
  Chain A of ricin
auauuccccaaacaauacccaauuauaaacuuuaccacagcgggugccacugugcaaagcuacacaaacuuuaucagagcuguucgcggucguuuaacaacuggagcugaugugagacaugaaauaccaguguugccaaacagaguugguuugccuauaaaccaacgguuuauuuuaguugaacucucaaaucaugcagagcuuucuguuacauuagcgcuggaugucaccaaugcauauguggucggcuaccgugcuggaaauagcgcauauuucuuucauccugacaaucaggaagaugcagaagcaaucacucaucuuuucacugauguucaaaaucgauauacauucgccuuuggugguaauuaugauagacuugaacaacuugcugguaaucugagagaaaauaucgaguugggaaaugguccacuagaggaggcuaucucagcgcuuuauuauuacaguacugguggcacucagcuuccaacucuggcucguuccuuuauaauuugcauccaaaugauuucagaagcagcaagauuccaauauauugagggagaaaugcgcacgagaauuagguacaaccggagaucugcaccagauccuagcguaauuacacuugagaauaguugggggagacuuucaacugcaauucaagagucuaaccaaggagccuuugcuaguccaauucaacugcaaagacguaaugguuccaaauucaguguguacgaugugaguauauuaaucccuaucauagcucucaugguguauagaugcgcaccuccaccaucgucacaguuuuaa

SEQ ID NO: 3 - RNA, 231 nt, Hepatitis C virus
acggggagcuaaacacuccaggccaauaggccauccuguuuuuuucccuuuuuuuuuuucuuuuuuuuuuuuuuuuuuuuuuuuuuuuuuuuuucuccuuuuuuuuuccucuuuuuuuccuuuucuuuccuuugguggcuccaucuuagcccuagucacggcuagcugugaaagguccgugagccgcuugacugcagagagugcugauacuggccucucugcagaucaagu

SEQ ID NO: 4 - RNA, 1433 nt, Artificial sequence
  Sequence of the  minimal  genomic RNA of positive polarity HCV
gccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacaccggauccauauuccccaaacaauacccaauuauaaacuuuaccacagcgggugccacugugcaaagcuacacaaacuuuaucagagcuguucgcggucguuuaacaacuggagcugaugugagacaugaaauaccaguguugccaaacagaguugguuugccuauaaaccaacgguuuauuuuaguugaacucucaaaucaugcagagcuuucuguuacauuagcgcuggaugucaccaaugcauauguggucggcuaccgugcuggaaauagcgcauauuucuuucauccugacaaucaggaagaugcagaagcaaucacucaucuuuucacugauguucaaaaucgauauacauucgccuuuggugguaauuaugauagacuugaacaacuugcugguaaucugagagaaaauaucgaguugggaaaugguccacuagaggaggcuaucucagcgcuuuauuauuacaguacugguggcacucagcuuccaacucuggcucguuccuuuauaauuugcauccaaaugauuucagaagcagcaagauuccaauauauugagggagaaaugcgcacgagaauuagguacaaccggagaucugcaccagauccuagcguaauuacacuugagaauaguugggggagacuuucaacugcaauucaagagucuaaccaaggagccuuugcuaguccaauucaacugcaaagacguaaugguuccaaauucaguguguacgaugugaguauauuaaucccuaucauagcucucaugguguauagaugcgcaccuccaccaucgucacaguuuuaaucuagaacggggagcuaaacacuccaggccaauaggccauccuguuuuuuucccuuuuuuuuuuucuuuuuuuuuuuuuuuuuuuuuuuuuuuuuuuuuucuccuuuuuuuuuccucuuuuuuuccuuuucuuuccuuugguggcuccaucuuagcccuagucacggcuagcugugaaagguccgugagccgcuugacugcagagagugcugauacuggccucucugcagaucaagu

SEQ ID NO: 5 - DNA, 36 bp, Artificial sequence
  PCR Primer NS3-Start
acacactggccaatggcgcccatcacggcctactcc

SEQ ID NO: 6 - DNA, 33 bp, Artificial sequence
  PCR Primer  NS5B-stop
gtgtgttctagatcatcggttggggagcaggta

SEQ ID NO: 7 - DNA, 36 bp, Artificial sequence
  PCR Primer NS5B2884S
tcattgaagggctccatggtcttagcgcatttacac

SEQ ID NO: 8 - DNA, 37 bp, Artificial sequence
  PCR Primer NS5B2884AS
taagaccatggagcccttcaatgatctgaggtaggtc

SEQ ID NO: 9 - DNA, 21 bp, Artificial sequence
  PCR Primer 5NC-Start
gccagcccccgattgggggcg

SEQ ID NO: 10 - DNA, 29 bp, Artificial sequence
  PCR Primer 5NC-Bam
ataggatccggtgttacgtttggtttttc

SEQ ID NO: 11 - DNA, 30 bp, Artificial sequence
  PCR Primer EGFP-Bam
tatggatccgtgagcaagggcgaggagctg

SEQ ID NO: 12 - DNA, 23 bp, Artificial sequence
  PCR Primer EGFP-Xba
cgctcagttggaattctagagtc

SEQ ID NO: 13 - DNA, 33 bp, Artificial sequence
  PCR Primer 3NC-Xba
atatattctagaacggggagctaaacactccag

SEQ ID NO: 14 - DNA, 22 bp, Artificial sequence
  PCR Primer 3NC-Stop
acttgatctgcagagaggccag

SEQ ID NO: 15 - DNA, 33 bp, Artificial sequence
  PCR Primer Hygro-Bam
atatatggatccaaaaagcctgaactcaccgcg

SEQ ID NO: 16 - DNA, 74 bp, Artificial sequence
  PCR Primer Hygro2A-Bam
atatatggatccgggcccagggttggactcgacgtctcccgcaagcttaagaagttcctttgccctcggacgag

SEQ ID NO: 17 - DNA, 40 bp, Artificial sequence
  PCR Primer T7-5UTR
taatacgactcactatagggccagccccctgatgggggcg

SEQ ID NO: 18 - DNA, 22 bp, Artificial sequence
  PCR Primer 3UTR-STOP
acttgatctgcagagaggccag

SEQ ID NO: 19 - DNA, 41 bp, Artificial sequence
  PCR Primer T7-3UTR
taatacgactcactataggacttgatctgcagagaggccag

SEQ ID NO: 20 - DNA, 21 bp, Artificial sequence
  PCR Primer 5UTR-Start
gccagcccccgattgggggcg

SEQ ID NO: 21 - DNA, 36 bp, Artificial sequence
  PCR Primer  Ric_Start
cacacaggatccatatttcccaaacaatacccaatc

SEQ ID NO: 22 - DNA, 33 bp, Artificial sequence
  PCR Primer Ric_stop
atatattctagattactaaaactgtgagctcgg

SEQ ID NO: 23 - DNA, 37 bp, Artificial Sequence
  PCR primer IFN-Bam
atatatggatccgccctgtccttttctttactgatgg

SEQ ID NO: 24 - DNA, 40 bp, Artificial Sequence
  PCR Primer IFN-Xba
atatattctagatcaatccttcctccttaatattttttgc

SEQ ID NO: 25 - DNA, 33 bp, Artificial Sequence
  PCR primer IRF1-Bam
atatatggatcccccatcactcggatgcgcatg

SEQ ID NO: 26 - DNA, 37 bp, Artificial Sequence; identical to SEQ ID NO: 23
  PCR primer IRF1-Xba

(19) United States
(12) Patent Application Publication
(10) Pub. No.: US 2011/0015255 A1
(43) Pub. Date: Jan. 20, 2011

(54) TREATMENT OF AN INFECTION BY AN RNA VIRUS DUE TO AN RNA DEPENDENT RNA POLYMERASE

(75) Inventors: Michel Ventura, Villenave D'Ornon (FR); Thérèse Astier-gin, Bordeaux (FR); Simon Litvak, Pessac (FR); Estelle Dumas, Bordeaux (FR)

Correspondence Address: HARNESS, DICKEY & PIERCE, P.L.C., P.O. BOX 828, BLOOMFIELD HILLS, MI, 48303, US

(73) Assignees: UNIVERSITÉ VICTOR SEGALEN - BORDEAUX 2, Bordeaux Cedex (FR); CENTRE NATIONAL DE LA RECHERCHE SCIENTIFIQUE -CNRS-, Paris Cedex 16 (FR)

(21) Appl. No.: 12/302,678

(22) PCT Filed: May 30, 2007
(86) PCT No.: PCT/FR2007/000899

§ 371 Date: Sep. 16, 2010

(30) Foreign Application Priority Data
May 30, 2006 (FR) 06/04806

(51) Int. Cl. A61K 31/7052; C07H 21/02; C12N 15/63; A61P 31/14

(52) U.S. Cl. 514/44 R; 536/23.1; 435/320.1

(57) ABSTRACT

The present invention concerns a simple RNA strand, which includes, from extremity 3′ to extremity 5′, (i) a sequence of complementary nucleic acid to the non-coding region 5′ (5′UTR) of the genomic RNA (strand (+)) of the hepatitis C virus (VHC) or another RNA virus replicating thanks to an RNA dependent RNA polymerase, the nucleic acid sequence of which allows the replication of said simple RNA strand molecule by the replication complex of said virus, (ii) the complementary nucleic acid sequence of a corresponding nucleic acid sequence from the site of internal entry of the ribosome (1RES), and (iii) the complementary nucleic acid sequence of a suicide gene or a coding gene for a protein interfering with the replication of the VHC virus or another RNA virus replicating thanks to an RNA dependent RNA polymerase; an ADN molecule allowing the transcription of said simple RNA strand molecule; a vector of nucleic acids including said molecules of nucleic acids; and a pharmaceutical compound including said molecules of nucleic acids or said vector of nucleic acid.

### CROSS-REFERENCE TO RELATED APPLICATIONS

This application is a National Phase Entry of International Application No. PCT/FR2007/000899, filed May 30, 2007, claiming priority to French Patent Application No. 06/04806, filed May 30, 2006, both of which are incorporated by reference herein.

### BACKGROUND AND SUMMARY

The present invention concerns a nucleic acid molecule, a pharmaceutical composition comprising such a nucleic acid molecule and the use of such a nucleic acid molecule for preparing a medication intended to treat or prevent an infection by an RNA virus replicating by virtue of an RNA-dependent RNA polymerase, and in particular an infection by the hepatitis C virus.

The hepatitis C virus (HCV) represents today a major problem in public health, in the light of the high prevalence of infection by it and the high risk of subsequent development towards chronic hepatitis of around 80%. This is because the hepatitis C virus (HCV) infects approximately 3% of the population and is responsible for approximately 170 million cases of chronic infections. The patients suffering from such a chronic infection are then liable to develop cirrhosis, with a risk estimated at 20%-30%, 20 years after the primary infection, which may develop into liver carcinoma. The primary infection generally being symptom free, the infection is diagnosed when the complications relating to chronic infection appear.

HCV is a positive-RNA virus, belonging to the family of Flaviviridae, discovered by the company Chiron in 1989 (Kuo et al, Science, vol 244(4902), p 362-4, 1989. Refined molecular analyses have demonstrated that there exist approximately 6 different genotypes of HCV, the said genotypes being subdivided into many subtypes. Concerning HCV infections, the majority of them are associated with genotype 1.

The genomic RNA of HCV contains a single open reading phase framed by 5′ non-coding sequences (5′UTR) and 3′ (3′UTR). If the 5′UTR sequence shows a high conservation whatever the genotype studied, the 3′UTR sequence for its part shows a high variability at its first 30 nucleotides according to the genotype studied. At the same time, the open reading phase codes according to the genotype considered for a polyprotein of 3008 to 3037 amino acids, which is cleaved co- and post-translationally by cellular and viral proteases in order to generate at least 10 mature viral proteins involved in the replication and morphogenesis of new virions. More specifically, the structural proteins are situated in the amino-terminal third of the said polyprotein and the non-structural proteins, some of which form the replication complex, in the carboxy-terminal part of the polyprotein.

The 5′UTR region contains a ribosome internal entry site that enables the viral genome ((+) strand), following the internalisation of the virion, to be translated in a cap-independent manner. After translation and maturation of the polyprotein, the replication complex is assembled, and then the RNA-dependent RNA polymerase (NS5B) commences the replication of the RNA. The replication complex of the HCV is therefore present within all the infected cells.

In more general terms, with the exception of the retroviridae that use a reverse transcriptase for their replication, viruses have an RNA genome replicating by virtue of an RNA-dependent RNA polymerase (RdRp) that synthesises, from genomic RNA, an RNA of opposite polarity serving as a replication intermediate. This intermediate replication RNA is in its turn copied by the RNA-dependent RNA polymerase in order to regenerate the genomic RNA.

This RdRp is coded by the virus and develops its activity within a replication complex, some proteins of which are also coded by the virus. Sequences carried by the viral genome are essential for the fixing of the replication complex that precedes the synthesis of the RNA strand opposed by the RdRp. This replication complex is fixed on the non-translated region situated at 3′ of the viral genome in order to synthesise the negative strand from the positive genome strand. Subsequently this replication complex is fixed on the non-translated region situated at 5′ of the negative strand in order to synthesise the positive genome strand. The replication complex of the virus having a RNA genome replicating by virtue of an RNA-dependent RNA polymerase is therefore present within all the infected cells.

The replication of the single-strand genomic RNA of the HCV requires an intermediate step corresponding to the synthesis of a “negative polarity anti-genome” strand ((−) strand), which will serve as a matrix for the synthesis of genomic RNAs of positive polarity ((+) strand). For this step of replication of the genomic RNA of the HCV, the two 5′UTR and 3′UTR sequences are essential. The 3′UTR sequence is necessary for the synthesis of the (−) strand from the (+) strand. At the same time, the 5′UTR sequence is necessary for the synthesis of the (+) strands of the new virions from the (−) strand.

The 5′UTR region has a length of around 340 nucleotides. This 5′UTR region comprises four domains having a structure of the stem/loop type (domains 5′UTR-dI to 5′UTR-dIV), the last domain 5′UTR-dIV overlapping the first nucleotides of the coding phase corresponding to the capsid protein of the polyprotein. This 5′UTR region is greatly conserved between the various strains of the HCV, both at the level of the nucleotide sequence and at a structural level. In addition to its role in the replication of the genome, the 5′UTR region is also involved in the initiation of the translation of the polyprotein. The minimum domain of the 5′UTR region for the replication to take place comprises the domains 5′UTR-dI and 5′UTR-dII (Friebe et al, J Virol, vol 75(24), p: 12047-57, 2001), the domain 5′UTR-dIII fulfilling a role of modulator thereon (Reusken et al, J Gen Virol, vol 84, p: 1761-69, 2003).

The minimum domain for the translation of the HCV corresponds to an internal entry site of the ribosome (IRES) to which the ribosome is directly fixed (Honda et al, Virology, vol 222(1), p: 31-42, 1996), which allows an initiation of the cap-independent translation. With the exception of the stem-loop constituting the 5′UTR-dI domain, the domains 5′UTR-dII to 5′UTR-dIV are necessary for the initiation of the translation. However, the location of the 3′ end of the IRES is debated. The assessment of the efficacy of initiation of the translation from segments of 5′ regions of the HCV genome with different lengths placed upstream of different reporter genes (SEAP, CAT) has led some authors to conclude that the 5′ part of the sequence coding the capsid protein C located directly downstream of the initiating AUG was necessary to obtain optimum efficacy of the IRES (Reynolds et al, RNA, vol 2(9), p: 867-78, 1996; Lu et al, Proc Natl Acad Sci USA, vol 93(4), p: 1412-7, 1996), which has been refuted by others (Rijnbrand et al, FEBS Lett, vol 365(2-3), p: 115-9, 1995).

One of the major difficulties in developing new antiviral treatments has stemmed from the absence of cell culture systems enabling the infection and replication of the HCV, but also the absence of small-sized animal models that would replace the chimpanzee, the only experimental animal model sensitive to infection by HCV. A first major advance has been able to be achieved with the development of bicistronic sub-genomic RNAs (replicons) of the HCV capable of replicating in the Huh-7 hepatocytary line (Lohmann et al, Science, vol 285(5424), p: 110-3, 1999). However, and despite the many improvements made to the system, no team has been able to show up to the present time that genomic replicons coding all the structural and non-structural proteins of the HCV are capable of generating viral particles. In addition, only replicons of genotype 1a, 1b and more recently genotype 2a (Kato et al, Gastroenterology, vol 125(6), p: 1808-17, 2003) have been able to be studied in this system. This cell system does however remain a model of predilection for studying viral replication and for developing novel and more effective antivirals.

Up to the present time, various treatments have been able to be developed for treating infections by HCV, the most effective current antiviral treatment consisting of a dual therapy based on the conjoint use of pegylated alpha interferon and a nucleoside analogue, ribavirin. However, the efficacy of this treatment seems to depend partly on the genotype of the virus responsible for the infection. This is because this therapy is effective only for approximately 40% to 50% of patients chronically infected by strains of the HCV of genotype 1 whereas it cures close on 80% of patients infected by HCVs of genotypes 2 or 3. Since genotype 1 is predominant in most of the world (60% to 90% of infections), the need to develop new antivirals and/or a vaccine constitutes an important need. Moreover, the use of interferon in this therapy makes it very expensive and often not well supported by the patient.

In order to improve the treatment of infections by HCV, much work has been carried out to discover new molecules that specifically inhibit steps of the viral cycle of the HCV. In the strategies developed, it is possible to distinguish (1) those that aim at targeting at the protein level specifically the viral enzymes that do not have a cellular homologue, in particular RNA-dependent RNA polymerase (NS5B) or the NS3 viral protease, and (2) the strategies that take the genome of the HCV as their target. In the context of this second strategy, many molecules have been developed for inhibiting the viral replication, such as in particular ribozymes, of the RNAi, aptamers or anti-sense nucleic acids. However, the compounds developed for these approaches have been confronted with the great genetic variability of the HCV, a consequence of the infidelity of the RNA polymerase of the HCV (NS5B) when the genome of the HCV is replicated. This is because, and since these molecules specifically target a sequence of the genome of the HCV, a single mutation within the viral genome may reduce or even cancel out their activity of inhibiting the replication of the HCV. In addition, the variability existing between the various genotypes of the HCV usually prevents the use of the same molecule for treating infections by different genotypes of the HCV.

There therefore exists at the present time a great need for identifying new molecules having a sufficiently broad specificity to target the many variants of the HCV, whilst keeping great efficacy vis-à-vis the different variants. Surprisingly, the inventors have now succeeded in developing a suicide vector in the form of a chimeric genomic RNA of positive polarity only in the cells infected by the HCV (cells expressing the HCV replication complex), the said chimeric genomic RNA of positive polarity allowing the translation of a toxic protein whose expression causes the death of the cells infected by the HCV. The negative-polarity chimeric genomic RNA according to the invention thus dispenses with the problems mentioned previously, in particular the variable efficacy of the treatments of the prior art according to the genotypes of the anti-HCV molecules and the great genetic variability at the basis of resistance to antivirals.

Thus a first object of the invention corresponds to a single-strand RNA molecule that comprises, starting from the 3′ end to the 5′ end:

(i) a complementary nucleic acid sequence of all or part of the 5′ non-coding region (5′UTR) of the RNA (+) strand of an RNA virus replicating by virtue of an RNA-dependent RNA polymerase, wherein said complementary nucleic acid sequence allows the replication of said single-strand RNA molecule through the replication complex of said virus;
(ii) possibly, the complementary nucleic acid sequence of a nucleic acid sequence corresponding to an internal entry site of the ribosome (IRES);
(iii) the complementary nucleic acid sequence of the nucleic acid sequence of a suicide gene or of a gene coding for a protein interfering with the replication of said virus; and
(iv) possibly a nucleic acid sequence complementary to the non-coding 3′ region (3′UTR) of the RNA of said virus.

The single-strand RNA molecule according to the invention is non-coding. Consequently the said single-strand RNA molecule, when it is present in cells not infected by the virus of interest in which the replication complex is therefore not expressed, does not make it possible to obtain the transcript of the suicide gene and its consequent translation and therefore to cause cell death. On the other hand, the single-strand RNA molecule according to the invention, when present in cells infected by the virus of interest that express the replication complex, is replicated as a complementary strand whose translation initiated at the IRES sequence causes synthesis of the protein of the suicide gene and consequently cell death.

Preferably, the RNA molecule according to the invention comprises a complementary nucleic acid sequence of a nucleic acid sequence corresponding to an internal entry site of the ribosome (IRES). However, this sequence is not obligatory for viruses not requiring an IRES for translation. A person skilled in the art will without difficulty be able to determine the viruses for which the RNA molecule according to the invention does not require a complementary nucleic acid sequence of a nucleic acid sequence corresponding to an internal entry site of the ribosome (IRES).

Preferably, for positive-RNA viruses, the RNA making it possible to obtain the complementary sequences (i) and (iv) is genomic RNA. “A positive-RNA virus” means a virus whose genomic RNA is directly coding. “Genomic RNA” means the RNA strand that corresponds to the RNA encapsidated in the viral particle.

In a preferred manner, a first object of the invention corresponds to a single-strand RNA molecule, corresponding to a negative-polarity chimeric genomic RNA ((−)RNA) of the hepatitis C virus (HCV), the said single-strand RNA molecule being characterised in that it comprises, starting from the 3′ end to the 5′ end:
(i) a nucleic acid sequence complementary to all or part of the non-coding 5′ region (5′UTR) of the genomic RNA ((+) strand) of the hepatitis C virus (HCV), wherein said complementary nucleic acid sequence allows replication of said single-strand RNA molecule through the replication complex of the HCV;
(ii) the complementary nucleic acid sequence of a nucleic acid sequence corresponding to an internal entry site of the ribosome (IRES);
(iii) the complementary nucleic acid sequence of the nucleic acid sequence of a suicide gene or of a gene coding for a protein interfering with the replication of the HCV virus; and
(iv) possibly a nucleic acid sequence complementary to the non-coding 3′ region (3′UTR) of the genomic RNA ((+) strand) of the HCV.

In a preferred manner, the RNA molecule according to the invention comprises a nucleic acid sequence complementary to the non-coding 5′ region (5′UTR) of the genomic RNA ((+) strand) of the hepatitis C virus (HCV), the said complementary nucleic acid sequence allowing replication of the said single-strand RNA molecule by the replication complex of the HCV. The single-strand RNA molecule according to the invention is non-coding. Consequently, the said single-strand RNA molecule, when present in cells not infected by the HVC in which the replication complex of the HVC is therefore not expressed, does not make it possible to obtain the transcript of the suicide gene and its consequent translation and therefore to cause cell death. On the other hand, the single-strand RNA molecule according to the invention, when present in cells infected by the HCV that express the replication complex, is replicated as a complementary strand ((+) strand) whose translation initiated at the IRES sequence causes synthesis of the protein of the suicide gene and consequently cell death.

The use of a single-strand RNA molecule corresponding to a negative-polarity chimeric genomic RNA ((−) strand) of the hepatitis C virus makes it possible, unlike a positive-polarity chimeric genomic RNA ((+) strand), to dispense with the replication step requiring recognition of the 3′UTR sequence of the (+) strand in order to obtain its replication as a (−) strand. Since the first 30 nucleotides of this 3′UTR region are more weakly conserved between the various genotypes compared with the 5′UTR region, the use of a positive-polarity chimeric genomic RNA, compared with a negative-polarity chimeric genomic RNA, could prove to be of very variable efficacy according to the genotype of the virus of the hepatitis C infecting the cell, tissue or patient to be treated.

Among the RNA viruses replicating by virtue of an RNA-dependent RNA polymerase, the following can be cited: the hepatitis C virus, as well as all the viruses belonging to the family of Flaviviridae, Cystoviridae, Birnaviridae, Reoviridea, Coronaviridae, Togaviridae, Arterivirus, Astroviridea, Caliciviridae, Picornaviridae, Potyviridae, Orthomyxoviridae, Filoviridae, Paramyxoviridae, Rhabdoviridae, Arenaviridea and Bunyaviridae. In a particularly preferred manner, the RNA viruses replicating by virtue of an RNA-dependent RNA polymerase are chosen from the group comprising: the dengue virus, the yellow fever virus, the West Nile virus and the bovine diarrhoea virus. Advantageously, the single-strand RNA molecule according to the invention can comprise ribonucleotides, modified or not, and preferably the said single-strand RNA molecule comprises non-modified ribonucleotides. Modified ribonucleotide means a natural ribonucleotide substituted by a synthetic analogue of a nucleotide, the said synthetic ribonucleotide analogue preferably being located at the 3′ or 5′ end of the nucleic acid molecule.

Synthetic analogues of preferred nucleotides are selected from the ribonucleotides having a sugar group or modified carbon group. Preferably the ribonucleotides having a modified sugar group have a 2′-OH group replaced by a group selected from a hydrogen atom, a halogen, an OR, R, SH, SR, NH₂, NHR, NR₂ or CN group, in which R is an alkyl, alkenyl or alkynyl group of 1 to 6 carbon atoms and the halogen is fluorine, chlorine, bromine or iodine. Preferably, the ribonucleotides having a modified carbon group have their phosphoester group bonded to the adjacent ribonucleotide, which is replaced by a modified group such as a phosphothioate group. However, it is also possible to use ribonucleotides having a modified purine or pyrimidine nucleus. As examples of such modified nuclei, uridines or cytadines modified in position 5 can be cited, such as 5-(2-amino)propyl uridine and 5-bromo uridine, adenosines and guanosines modified in position 8, such as 8-bromo quanosine, deazoted nucleotides such as 7-deaza-adenosine, N- and O-alkylated nucleotides, such as N6-methyl adenosine. These various modifications may also be combined.

“A nucleic acid sequence complementary to the non-coding 5′ region (5′UTR) of the genomic RNA ((+) strand) of the HCV, the said complementary nucleic acid sequence allowing replication of the said single-strand RNA by the replication complex of the HCV” means a nucleic acid sequence comprising at least the complementary sequence of a sequence comprising the stem-loop domains I and II (5′UTR-dI and 5′UTR-dII) of the said 5′UTR region, preferably at least the complementary sequence of a sequence comprising the stem-loop domains I, II and III (5′UTR-dI, 5′UTR-dII and 5′UTR-dIII) of the said 5′UTR region. A person skilled in the art, in the light of his general knowledge, will be able to simply determine the sequence of the various stem-loop domains in the 5′UTR region of the HCV virus of a given genotype, or even of a given subtype. The general knowledge of a person skilled in the art concerning the HCV and the positioning of these domains are in particular illustrated on the site http://euhcvdb.ibcp.fr/euHCVdv/.

By way of example, the 5′UTR-dl domain goes from the nucleotides 5 to 20 of the sequences having the accession number M62321, M67463 or AF009606 for the genotype 1a; 1 to 8 of the sequence having the accession number D90208 or 1 to 11 of the sequence having the accession number M58335 for the genotype 1b; 5 to 20 of the sequence having the accession number D14853 or AY051292 for the genotype 1c; 5 to 19 of the sequence having the accession number D00944 or AB047639 for the genotype 2a; 5 to 20 of the sequence having the accession number D10988 or AB030907 for the genotype 2b; 5 to 19 of the sequence having the accession number D50409 for the genotype 2c; 6 to 20 of the sequence having the accession number AB031663 for the genotype 2k; 5 to 18 of the sequence having the accession number D17763 or D28917 for the genotype 3a; 5 to 18 of the sequence having the accession number D49374 for the genotype 3b; 5 to 18 of the sequence having the accession number D63821 for the genotype 3k; 5 to 19 of the sequence having the accession number D84262 for the genotype 6b; 5 to 18 of the sequence having the accession number D84263 for the genotype 6d; 5 to 18 of the sequence having the accession number D63822 for the genotype 6g, 5 to 18 of the sequence having the accession number D84265 for the genotype 6h; 5 to 18 of the sequence having the accession number D84264 for the genotype 6k.

By way of example, the domain 5′UTR-dII goes from the nucleotides 44 to 118 of the sequences having the accession number M62321, M67463 or AF009606 for the genotype 1a; 35 to 109 of the sequence having the accession number M58335 or 32 to 106 of the sequence having the accession number D90208 for the genotype 1b; 44 to 118 of the sequences having the accession number D14853 or AY051292 for the genotype 1c; 43 to 117 of the sequences having the accession number D00944 or AB047639 for the genotype 2a; 44 to 118 of the sequences having the accession number AB030907 or D10988 for the genotype 2b; 43 to 117 of the sequence having the accession number D50409 for the genotype 2c; 44 to 118 of the sequence having the accession number AB031663 for the genotype 2k; 42 to 116 of the sequence having the accession number D17763 or D28917 for the genotype 3a; 42 to 116 of the sequence having the accession number D49374 for the genotype 3b; 42 to 116 of the sequence having the accession number D63821 for the genotype 3k; 1 to 72 of the sequence having the accession number AY859526 for the genotype 6a; 42 to 116 of the sequence having the accession number D84262 for the genotype 6b; 41 to 115 of the sequence having the accession number D84263 for the genotype 6d; 41 to 115 of the sequence having the accession number D63882 for the genotype 6g; 41 to 115 of the sequence having the accession number D84265 for the genotype 6h; 41 to 115 of the sequence having the accession number D84264 for the genotype 6k.

By way of example, the domain 5′UTR-dIII goes from the nucleotides 125 to 323 of the sequences having the accession number M62321, M67463 or AF009606 for the genotype 1a; 116 to 314 of the sequence having the accession number M58335 or 113 to 311 of the sequence having the accession number D90208 for the genotype 1b; 125 to 323 of the sequences having the accession number D14853 or AY051292 for the genotype 1c; 124 to 322 of the sequences having the accession number D00944 or AB047639 for the genotype 2a; 125 to 323 of the sequences having the accession number AB030907 or D10988 for the genotype 2b; 124 to 322 of the sequence having the accession number D50409 for the genotype 2c; 125 to 323 of the sequence having the accession number AB031663 for the genotype 2k; 123 to 321 of the sequences having the accession number D17763 or D28917 for the genotype 3a; 123 to 321 of the sequence having the accession number D49374 for the genotype 3b; 123 to 321 of the sequence having the accession number D63821 for the genotype 3k; 63 to 261 of the sequence having the accession number Y11604 for the genotype 4a; 30 to 228 of the sequence having the accession number AF064490 for the genotype 5a; 79 to 277 of the sequence having the accession number AY85926 for the genotype 6a; 123 to 324 of the sequence having the accession number D84262 for the genotype 6b; 122 to 320 of the sequence having the accession number D84263 for the genotype 6d; 122 to 320 of the sequence having the accession number D63822 for the genotype 6g; 122 to 320 of the sequence having the accession number D84265 for the genotype 6h; 122 to 320 of the sequence having the accession number D84264 for the genotype 6k.

Advantageously, “a nucleic acid sequence complementary to the non-coding 5′ region (5′UTR) of the genomic RNA ((+) strand) of the HCV, the said complementary nucleic acid sequence allowing replication of the said single-strand RNA molecule by the replication complex of the HVC” means the complementary nucleic acid sequence of the sequence going from position 1 to 120 of the genomic RNA ((+) strand) of the HCV, preferably comprising going from position 1 to 150 and particularly preferably going from position 1 to 341 of the genomic RNA ((+) strand) of the HCV. Position 1 corresponds to the first nucleotide of the genomic RNA ((+) strand) of a complete hepatitis C virus (HCV). 5′UTR region of the genomic RNA ((+) strand) of the HCV means the 5′UTR region of the genomic RNA of a hepatitis C virus (HCV) of genotype 1, 2, 3, 4, 5 or 6, or a sequence derived therefrom. Derived sequence means a sequence having at least 80% identity with the sequence of the 5′UTR region of the genomic RNA of a hepatitis C virus of genotype 1, 2, 3, 4, 5 or 6, preferably at least 85%, especially at least 90%, and particularly preferably at least 95% identity. Preferably, the sequence of the 5′UTR region of the genomic RNA of HCV corresponds to the sequence of the 5′UTR region of the genome of the genomic RNA of a hepatitis C virus (HCV) of genotype 1.

In a particularly preferred manner, the sequence of the 5′UTR region of the genomic RNA of the HCV corresponds to the sequence going from position 1 to 120 of the sequence SEQ ID No: 1, preferably going from position 1 to 150 and particularly preferably going from position 1 to 341 of the sequence SEQ ID No: 1 or a sequence derived therefrom. A person skilled in the art will be able to identify, without difficulty and in the light of his general knowledge, a nucleic acid sequence corresponding to an internal entry site of the ribosome (IRES). Such an IRES sequence may be of eukaryotic origin or viral origin.

By way of example of an IRES sequence of eukaryotic origin, the IRES sequence of the transcripts coding for the proteins chosen from the group comprising the FGF (Fibroblast Growth Factor) family, the connexine family, the cyclin-dependent kinase-inhibiting protein p27, BCL 2, HSP 101, HSP 70, the proto-oncogenes c-myc, L-myc, n-nyc or the Mnt transcription repressor can be cited. By way of example of an IRES sequence of viral origin, the IRES sequence of the poliomyelitis virus, of the encephalomyocarditis virus (EMCV), of the GBV-A virus, of the GBV-B virus, of the GBV-C virus, of the hepatitis C virus, of the bovine viral diarrhoea virus (BVDV), of the A and B viruses of equine rhinitis (ERAV and ERBV), of the ZAM, Idefix and gypsy retroelements or of HIV can be cited. Preferably, the IRES sequence is of viral origin and particularly preferably the said IRES sequence corresponds to the IRES sequence of the HCV, which corresponds to the stem-loop domains II to IV (5′UTR-dII to 5′UTR-dIV) of the 5′UTR region of the genomic RNA ((+) strand) of the HCV.

By way of example, the IRES sequence of the HCV goes from nucleotides 44 to 354 of the sequences having the accession number M62321, M67463 or AF009606 for the genotype 1a; 35 to 345 of the sequence having the accession number M58335 or 32 to 342 of the sequence having the accession number D90208 for the genotype 1b; 44 to 345 of the sequences having the accession number D14853 or AY051292 for the genotype 1c; 43 to 353 of the sequences having the accession number D00944 or AB047639 for the genotype 2a; 44 to 354 of the sequences having the accession number AB030907 or D10988 for the genotype 2b; 43 to 353 of the sequence having the accession number D50409 for the genotype 2c; 44 to 354 of the sequence having the accession number AB031663 for the genotype 2k; 42 to 352 of the sequences having the accession number D17763 or D28917 for the genotype 3a; 42 to 352 of the sequence having the accession number D49374 for the genotype 3b; 42 to 352 of the sequence having the accession number D63821 for the genotype 3k; 1 to 308 of the sequence having the accession number AY859526 for the genotype 6a; 42 to 355 of the sequence having the accession number D84262 for the genotype 6b; 41 to 351 of the sequence having the accession number D84263 for the genotype 6d; 41 to 351 of the sequence having the accession number D63822 for the genotype 6g; 41 to 351 of the sequence having the accession number D84265 for the genotype 6h; 41 to 351 of the sequence having the accession number D84264 for the genotype 6k.

Advantageously, “IRES sequence of the HCV” means the sequence going from position 30 to 355 of the genomic RNA ((+) strand) of the HCV, preferably going from position 25 to 370 and particularly preferably going from position 20 to 385 of the genomic RMA ((+) strand) of the HCV. IRES sequence of the HCV means the IRES sequence of a genomic RNA ((+) strand) of a hepatitis C virus (HCV) of genotype 1, 2, 3, 4, 5 or 6, or a sequence derived therefrom, preferably the IRES sequence of the genomic RNA ((+) strand) of a hepatitis C virus (HCV) of genotype 1. Derived sequence means a sequence having an identity of at least 80% with the sequence of the IRES sequence of the genomic RNA ((+) strand) of a hepatitis C virus (HCV) of genotype 1, 2, 3, 4, 5 or 6, preferably at least 85%, especially at least 90%, and particularly preferably at least 95% identity. In a particularly preferred manner, the IRES sequence of the HCV corresponds to the sequence going from position 30 to 355 of the sequence SEQ ID No: 1, preferably going from position 25 to 370 and in a particularly preferred manner going from position 20 to 386 of the sequence SEQ ID No: 1 or a sequence derived therefrom.

According to a preferred embodiment, the single-strand RNA molecule according to the invention comprises the complementary sequence of the sequence going from position 1 to 355 of the genomic RNA ((+) strand) of the HCV, preferably going from position 1 to 370 and in a particularly preferred manner going from position 1 to 385 of the genomic RNA ((+) strand) of the HCV. Sequence of the genomic RNA ((+) strand) of the HCV means the sequence of a hepatitis C virus (HCV) of genotype 1, 2, 3, 4, 5 or 6, or a sequence derived therefrom, and preferably means the sequence of a hepatitis C virus (HCV) of genotype 1. Derived sequence means a sequence having at least 80% identity with the sequence of the genomic RNA ((+) strand) of a hepatitis C virus (HCV) of genotype 1, 2, 3, 4, 5 or 6, preferably at least 85%, especially at least 90%, and in a particularly preferred manner at least 95% identity.

Preferably, the single-strand RNA molecule according to the invention comprises a complementary sequence of the sequence going from position 1 to 355 of the sequence SEQ ID No: 1, preferably going from position 1 to 370 and in a particularly preferred manner going from position 1 to 386 of the sequence SEQ ID No: 1 or a sequence derived therefrom. A person skilled in the art will be able to determine, without difficulty and in the light of his general knowledge, the nucleic acid sequences of the suicide genes than can be used for the molecule according to the invention.

A person skilled in the art will be able to identify simply the genes coding from proteins interfering with the replication of the HCV virus in the light of his general knowledge. By way of example of proteins interfering with the replication of the HCV, α interferon, β interferon and γ interferon can be cited, preferably a interferon and particularly preferably a 2a or a 2b interferon. By way of example of genes coding for proteins interfering with the replication of an RNA virus replicating by virtue of an RNA-dependent RNA polymerase, in particular the hepatitis C virus, the genes coding for an interferon regulating factor (IRF) can also be cited.

Suicide gene means a gene that codes for a protein product that causes death of the cell in the presence or absence of drugs. According to a particular embodiment, the suicide gene codes for a bacterial or viral enzyme causing the death of the cell in the presence of a specific “drug”. More specifically, the said enzymes convert the inactive form of a drug (prodrug) present in the environment into its active toxic form causing death of the cell, for example by an inhibition of the synthesis of nucleic acid. A person skilled in the art will be able to identify, without difficulty and in the light solely of his general knowledge, the suitable suicide genes.

By way of example of such suicide genes, the genes of HSV thymidine kinase (HSVItk) or cytosine deaminase (CD), used respectively with Ganciclovir or 5-FU respectively (cf international application PCT WO 2005/092374, pages 11 and 12), can be cited. According to another particular embodiment, the suicide gene codes for a protein toxin causing death of the cell. By way of proteins that can be used, bacterial exotoxins, fungal toxins, toxins of eukaryotic origin, vegetable origin or viral origin or proteins derived therefrom can be cited. Derived protein means a protein having at least 80% identity with a bacterial exotoxin, a fungal toxin, a toxin of eukaryotic origin, vegetable origin or viral origin, preferably at least 85%, especially at least 90%, and in a particularly preferred manner at least 95% identity.

Advantageously again, the protein toxin causing death of the cell belongs to the RIP (Ribosome-Inactivation Protein) family, the said protein toxins existing in many species of plants, bacteria or fungi (Van Damme et al, Crit. Rev Plant Sci, vol 20, p: 395-465, 2001; Sandvig and Van Deurs, FEBS Lett, vol 529, p: 49-53, 2002). The proteins in the RIP family are similar to the lectin family, but their toxicity is much greater than the latter. The proteins of the RIP family are divided into two types according to their molecular structure.

Type I contains the proteins that comprise a single polypeptide chain of approximately 30 kDa and are devoid of lectin activity allowing fixing to the cell surface, which confers reliable toxicity on them. Type II contains the proteins comprising two polypeptide chains A and B with distinct properties. The haptomer or chain B (Binding), which contains a lectin domain, interacts with a sugar or a glycosylated compound on the surface of the cell and facilitates entry of chain A in the cell. The effectomer, or chain A (Activity), carries the toxic activity. The best known of these toxins is ricin (from Ricinus communis), but other plant toxins such as abrin, (from Abrus precatorius), modeccin (from Adenai digitata), volkensin (from Adenai volkensii) and viscumin (from Viscum album), have the same properties. There also come within this group certain bacterial toxins such as Shiga toxin produced by Shigella dysentria and similar toxins (Shiga-like toxins or SLTs) secreted by certain strains of Escherichia coli (STEC, also called VTEC since they are cytotoxic for Vero cells), but also Citrobactor freundii, Aeromonas hydrophila, Aeromonas civiae and Enterobactor cloacae. The RIPs of type 2 are much more effective that RIPs of type 1; this is because, although powerful inhibitors of protein synthesis in acellular preparations, the type-1 RIPs are much less toxic in mice (DL50 from 1 to 40 mg/kg) than type-2 RIPs (DL50 of ricin: 2 μg/kg).

Advantageously, the suicide gene codes for a protein toxin in the type-2 RIP family or a derived protein, preferably for chain A of a protein toxin of the type-2 RIP family such as ricin, abrin, modeccin, volkensin, viscumin and the Shiga toxin, and in a particularly preferred manner for chain A of the ricin. In a particular preferred manner, the suicide gene corresponds to the sequence SEQ ID No: 2 or to a derived sequence. Derived sequence means a sequence having at least 80% identity with the sequence SEQ ID No: 2, preferably at least 85%, especially at least 90%, and in a particularly preferred manner at least 95% identity.

A persons skilled in the art, in the light of his general knowledge, will be able to simply determine the complementary nucleic acid sequence of the non-coding 3′ region (3′UTR) of the genomic RNA ((+) strand) of a hepatitis C virus (HCV) of a given genotype, or even of a given subtype. The general knowledge of a person skilled in the art concerning HCV and the positioning of this region is in particular illustrated on the site http://euhcvdb.ibcp.fr/euHCVdb/.

By way of example, the 3′UTR region goes from nucleotides 9378 to 9646 in the sequence having the accession number AF009606 for the genotype 1a; 9443 to 9678 of the sequence having the accession number AB047639 for the genotype 2a; 9444 to 9654 of the sequence having the accession number AB030907 for the genotype 2b; 9403 to 9628 of the sequence having the accession number D84262 for the genotype 6b; 9381 to 9615 of the sequence having the accession number D84263 for the genotype 6d; 9387 to 9621 of the sequence having the accession number D84264 for the genotype 6k. 3′UTR region of the genomic RNA ((+) strand) of the HCV means the 3′UTR region of the genomic RNA of a hepatitis C virus (HCV) of genotype 1, 2, 3, 4, 5 or 6, or a derived sequence thereof, preferably of a hepatitis C virus of genotype 1. Derived sequence means a sequence having at least 80% identity with the sequence of the 3′UTR region of the genomic RNA of a hepatitis C virus of genotype 1, 2, 3, 4, 5 or 6, preferably at least 85%, in particular at least 90%, and in a particularly preferred manner at least 95% identity. Preferably, the sequence of the 3′UTR region of the genomic RNA of the HCV corresponds to the sequence SEQ ID No: 3 or a sequence derived therefrom.

The single-strand RNA molecule according to the invention can be obtained by chemical synthesis methods or by molecular biology methods, in particular by transcription using DNA matrices or plasmids isolated from recombinant microorganisms. Preferably, this transcription step uses phage RNA polymerases such as RNA polymerase T7, T3 or SP6.

According to a second preferred embodiment, the single-strand RNA molecule according to the invention corresponds to the complementary sequence of the sequence SEQ ID No: 4 or a derived sequence. Derived sequence means a sequence having at least 80% identity with the sequence SEQ ID No: 4, preferably at least 85%, especially at least 90%, and in a particularly preferred manner at least 95% identity.

A second object of the invention corresponds to a DNA molecule, preferably double-strand DNA, allowing transcription of the single-strand RNA molecule described previously. Advantageously, the said DNA molecule comprises a nucleic acid sequence complementary to the nucleic acid sequence of the single-strand RNA molecule described previously, the third complementary nucleic acid sequence being bonded operationally to a nucleic acid sequence allowing its transcription in a eukaryotic or prokaryotic cell, preferably eukaryotic, and therefore the obtaining of the single-strand RNA molecule described previously. The said DNA molecule therefore makes it possible to obtain the single-strand RNA molecule corresponding to a negative-polarity chimeric genomic RNA directly and without passing through the successive steps of transcription of a positive-polarity chimeric genomic RNA and replication thereof.

A nucleic acid sequence allowing transcription means any transcription regulation sequence, such as a promoting or activating sequence, preferably a promoting sequence. The said promoting sequence may correspond, for example, to a cellular or viral promoter, and to a constituent or inducible promoter. By way of example of promoters constituting mammals, the promoters of the following genes can be cited non-limitatively: hypoxanthine, phosphoribosyl transferase (HPTR), adenosine deaminase, pyruvate kinase, β-actin, muscle creatine kinase and human elongation factor. By way of example of viral promoters having a constituent expression in the cells of mammals, there can be cited, non-limitatively, the following virus promoters: SV40, papilloma virus, adenovirus, human immunodeficiency virus (HIV), cytomegalovirus (CMV), Rous sarcoma virus (RSV) and hepatitis B virus (HBV). Other constituent promoters are well known to persons skilled in the art. Useful promoting sequences also include inducible promoters and those permitting transcription consequent upon the addition of an inducible agent. By way of example, the promoters of genes in the metallothionine family whose transcription is induced in the presence of certain metal ions can be cited. Other inducible promoters can be simply identified by persons skilled in the art in the light of their general knowledge. In general, the promoting sequence includes non-transcribed 5′ sequences involved in the initiation of the transcription such as a TATA box.

Another object of the invention consists of a nucleic acid vector comprising a nucleic acid molecule as described previously, in particular an RNA or DNA molecule. “Nucleic acid vector” means any support for facilitating the transfer of the said RNA or DNA molecules into the cells, preferably into the cells potentially infected by the hepatitis C virus. Preferably the vector according to the invention makes it possible to transport the said nucleic acid molecules while limiting the degradation thereof with respect to their transportation in the absence of a vector. The vector possible comprises the promoting sequences described previously. By way of example of vectors that can be used, plasmids, phagemides, viruses and other derived vectors of viral or bacterial origin can be cited non-limitatively. Preferred viral vectors include adenoviruses (modified), which are capably of infecting a very large number of species and cell types, lentiviruses (modified in particular by a modification of the envelope protein so as to obtain the required tropism) or hepatitis A and B viruses (modified) which are capable of specifically infecting the hepatic cells. Preferred non-viral vectors include plasmid vectors, which are described extensively in the prior art (see in particular Sanbrook et al, “Molecular Cloning: A Laboratory Manual”, Second Edition, Cold Spring Harbor Laboratory Press, 1989). The plasmids can be administered by many methods, in particular topical or parenteral. For example, the plasmids can be injected by intramuscular, intradermal, intrahepatic or subcutaneous method. The nucleic acid vector according to the invention can comprises active selection markers in the eukaryotic and/or prokaryotic cells.

Another object of the invention consists of a pharmaceutical composition comprising a nucleic acid molecule, RNA or DNA, or a vector as described previously. Advantageously, the said pharmaceutical composition comprises a pharmaceutically acceptable support. By way of pharmaceutically acceptable support, the compositions according to the invention can comprise emulsions, microemulsions, oil-in-water or water-in-oil emulsions, or other types of emulsion. The composition can also comprise one or more additives, such as diluents, excipients or stabilisers (see in particular Ullman's Encyclopaedia of Industrial Chemistry, 6th Ed, 1989-1998, Marcel Dekker; Ansel et al, Pharmaceutical Dosage Forms and Drug Delivery Systems, Williams & Wilkins, 1994). The composition can comprise water or a solubilisation buffer, the said buffers including, non-limitatively, phosphate buffer saline (PBS), phosphate buffer saline without Ca++/Mg++, physiological serum (150 mM NaCl in water) or tris buffers.

Another object of the invention consists of the use of a nucleic acid molecule or a vector as described previously for preparing a pharmaceutical composition intended to prevent or treat an RNA virus replicating by virtue of an RNA-dependent RNA polymerase in a patient. Another object of the invention consists of using a nucleic acid molecule or a vector as described previously for preparing a pharmaceutical composition intended to prevent or treat an infection by the hepatitis C virus (HCV) in a patient. “Patient” means a mammal, preferably a human.

The composition according to the invention can be administered topically or parenterally in a therapeutically effective quantity, in particular by intramuscular, intradermal, intrahepatic, or subcutaneous injection, preferably by intrahepatic injection. The administration of the composition according to the invention can be effected by gene transfer methods known to persons skilled in the art. Common gene transfer methods include calcium phosphate, DEAE-Dextran, electroporation, microinjection, viral methods and cationic liposomes (Graham and Van Der Eb, Virol, vol 52, p: 456, 1973; McCuthan and Pagano, J Natl Cancer Inst, Vol 41, p: 351, 1968; Chu et al, Nucl Acids Res, vol 15, p: 1311; Fraley et al, J Biol Chem, vol 255, p: 10431, 1980; Capecchi et al, Cell, vol 22, p: 479, 1980, Feigner et al, Proc Natl Acad Sci USA, vol 84, p: 7413, 1988).

An effective quantity of nucleic acid molecules to be administered to a patient can be determined simply by a person skilled in the art. By way of example, an effective quantity of nucleic acid molecules is between 0.001 mg and 10 g/kg of the patient to be treated, preferably between 0.01 mg and 1 g/kg, and in a particular preferred manner between 0.1 and 100 mg/kg. Other characteristics of the invention will emerge in the following examples, without for all that these constituting any limitation on the invention.

### EXAMPLES

### 1) Construction of a Cell System Allowing Expression of Genomic RNA Derived from HCV

In order to analyse the replicative potential of chimeric genomic RNA according to the invention, a cell system that constituently synthesises the replication complex of HCV has been developed by inserting the non-structural proteins of HCV (NS3-NS5B) in the genome of the cells of the Huh7 hepatoma cell line. For this purpose, a vector pcDNA3.1/NS3-5B(2884R/G) coding for the said non-structural proteins was constructed as follows:
The genes coding for the non-structural proteins covering NS3 to NS5B were amplified using the infectious isolate J4L6 of the HCV, a strain of the genotype 1a (Yanagi et al, Virology, vol 244(2), p: 161-72, 1998) using the polymerase Herculase® (STRATEGENE) and the primers NS3-Start (SEQ ID No 5: ACACACTGGCCAATGGCGCCCATCACGGCCTACTCC) and NS5B-stop (SEQ ID No 6: GTGTGTTCTAGATCATCGGTTGGGGAGCAGGTA).
The fragment NS3-5B was then inserted between the sites EcoRV and XbaI of the plasmid pcDNA3.1 (INVITROGEN), thus producing the vector pcDNA3.1/NS3-5B.
A mutation of the residue Gly into Arg at position 2884 (Pietschmann et al) was then introduced by direct mutageneses using the primers NS5B2884S (SEQ ID No 7: TCATTGAAGGGCTCCATGGTCTTAGCGCATTTACAC) and NS5B2884AS (SEQ ID No 8: TAAGACCATGGAGCCCTTCAATGATCTGAGGTAGGTC) and the DNA polymerase Taq Phusion® (OZYME). The PCR reaction was performed on the vector pcDNA3.3/NS3-5B, the product PCR obtained was then digested by the enzyme Dpn 1 (PROMEGA) and directly amplified in a culture of E coli DH5α bacteria. Finally, the plasmid pcDNA3.1/NS3-5B(2884R/G) containing the mutated sequence was selected by sequencing.

A million Huh7 cells were then transfected by 2.5 μg of vector plasmid pcDNA3.1/NS3-5B(2884R/G) using Lipofectin® with reagent+(INVITROGEN) added in accordance with the instructions of the supplier. The cells were then cultivated in modified Dulbecco medium supplemented with 10% foetal calf serum (FCS; inactivated by heat) and the antibiotic G418 (1 mg/ml) at 37° C. in a 5% CO₂ atmosphere. The expression of the plasmid pcDNA3.1/NS3-5B(2884R/G) was then analysed by the Western Blot technique in the various clones resistant to the G418 antibiotic (Huh7/NS3-5B). The results showed that the NS5B protein was constituently expressed and had the expected molecular weight, indicating that the NS3-5B polyprotein was correctly matured by the NS3 protease.

### 2) Capacity for Replication of a Negative-Polarity Minimal Genomic RNA Derived from the HCV in the Cell Line Huh7/NS3-5B

2-1: Construction of a Negative-Polarity Minimal Genomic RNA Derived from HCV:

In order to evaluate the replication activity of the cell line Huh7/NS3-5B a minimal replication sequence named 5UTR-H2AE-3UTR was constructed. This sequence 5UTR-H2AE-3UTR was formed from the 5′UTR region of the HCV, a sequence coding for a polyprotein consisting of the sequences of the hygromycin resistance protein, the 2A protein of the aphthous fever virus (FMDV) and the EGFP protein, followed by the 3′NC region of the HCV, was constructed.

The construction of the vector coding for the sequence 5UTR-H2AE-3UTR was effected in two steps:

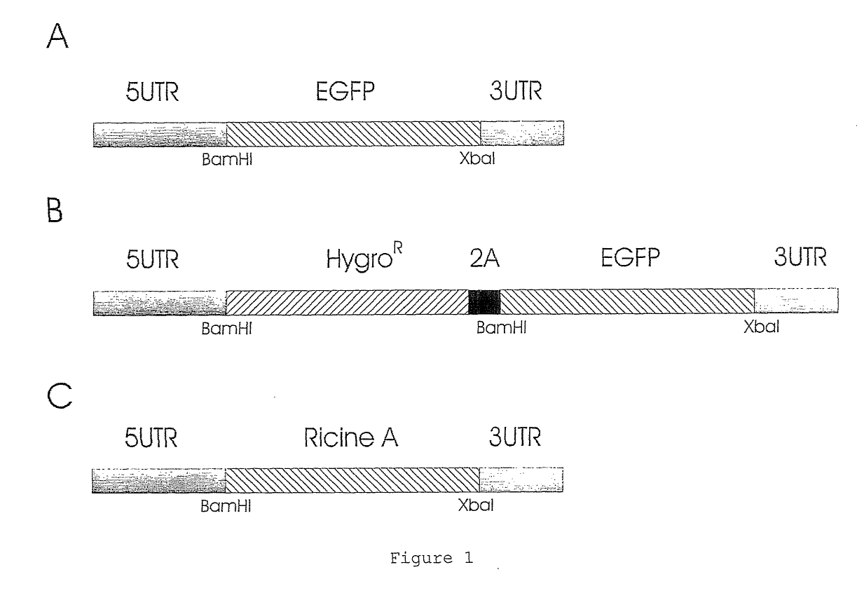

Step 1: The construction of the vector plasmid pGEM-T/5UTR-EGFP-3UTR (FIG. 1A) was effected by amplification of the 5′UTR and 3′UTR regions (or non-coding region), coming respectively from the isolate coni and the strain H77, and the gene of the EGFP (enhanced green fluorescent protein). The 5′NC sequence, extended to the first 27 nucleotides coding for the capsid protein (C protein), was amplified by means of the primers 5NC-Start (SEQ ID No 9: GCCAGCCCCCGATTGGGGGCG) and 5NC-Bam (SEQ ID No 10: ATAGGATCCGGTGTTACGTTTGGTTTTTC) of the coni isolate as a matrix (EMBL X61596), the sequence EGFP was amplified by the polymerase Taq Gold® (ROCHES) by means of the primers EGFP-Bam (SEQ ID No 11: TATGGATCCGTGAGCAAGGGCGAGGAGCTG) and EGFP-Xba (SEQ ID No 12: CGCTCAGTTGGAATTCTAGAGTC). The fragments obtained were then cleaved by the restriction enzyme Bam HI, ligatured by means of the ligase T4 DNA (PROMEGA) at 16° C. during the night, and then amplified once again with the primers 5NC-Start (SEQ ID No: 9) and EGFP-Xba (SEQ ID No: 12), thus generating the fragment 5UTR-EGFP. The 3′NC region was obtained by polymerase chain reaction (PCR) using the primers 3NC-Xba (SEQ ID No 13: ATATATTCTAGAACGGGGAGCTAAACACTCCAG) and 3NC-stop (SEQ ID No 14: ACTTGATCTGCAGAGAGGCCAG) of the strain VHC H77 (EMLB AF011753). The amplification product and the 5UTR-EGFP fragment were digested by the enzyme XbaI and ligatured under the conditions described above, and the ligation product obtained was amplified using the primers 5NC-Start and 3NC-stop. Finally the resulting fragment was inserted in the vector plasmid pGEM-T PROMEGA) producing the vector pGEM-T/5UTR-EGFP-3UTR. The sequence of the vector pGEM-T/5NC-EGFP-3NC obtained was confirmed by sequencing.
Step 2: The construction of the plasmid pGEM-T/5UTR-H2AE-3UTR (see FIG. 1B) was effected by inserting the sequence of hygromycin phosphotransferase (HygroR) and the sequence coding for the 2A protein of aphthous fever virus (FMDV, standing for Foot and Mouth Disease Virus) between the 5′UTR region and the EGFP sequence of pGEM-T/5UTR-EGFP-3UTR.

The hygromycin sequence was obtained by an amplification by PCR of the vector plasmid psiSTRIKE™ (PROMEGA) using the primers Hygro-Bam (SEQ ID No 15: ATATATGGATCCAAAAAGCCTGAACTCACCGCG) and Hygro2A-Bam (SEQ ID No 16: ATATATGGATCCGGGCCCAGGGTTGGACTCGACGTCTCCCGCAAGCTTA AGAAGTTCCTTTGCCCTCGGACGAG); the said primer Hygro2A-Bam comprising the 42 nucleotides of the protein 2A sequence. The amplification product obtained was then inserted at the BamHI site of the plasmid pGEM-T/5UTR-EGFP-3UTR, in order to obtain the vector pGEM-T/5UTR-H2AE-3UTR. Finally, the sequence of the vector pGEM-T/5UTR-H2AE-3UTR obtained was confirmed by sequencing.

The promoter of the phage T7 necessary for the transcription was introduced by PCR. In order to transcribe the (+) strand the primers

T7-5UTR (SEQ ID No 17: TAATACGACTCACTATA GGGCCAGCCCCCTGATGGGGGCG) and 3UTR-STOP (SEQ ID No 18: ACTTGATCTGCAGAGAGGCCAG) were used. In order to transcribe the (−) strand the primers T7-3UTR (SEQ ID No 19: TAATACGACTCACTATAGGACTTGATCTGCAGAGAGGCCAG) and 5UTR-Start (SEQ ID No 20: GCCAGCCCCCGATTGGGGGCG) were used. Finally, the minimal genomic RNA of negative polarity was obtained by transcription of 1 μg of PCR product using polymerase T7 (PROMEGA) following the instructions of the manufacturer. Finally the RNAs were purified by means of an RNeasy® kit (QIAGEN) following the instructions of the manufacturer.

2-2: Replication of the Negative-Polarity Derived Minimal Genomic RNA of HCV in the Cell Line Huh7/NS3-58:

24-well culture plates were seeded at the rate of 10⁵ Huh7/NS3-5B cells per well and incubated for 24 hours at 37° C. The cells obtained were then transfected using 1 μg of positive-polarity 5UTR-H2AE-3UTR transcripts mixed with 3 μl of DMRIE-C (INVITROGEN) in accordance with the recommendations of the supplier. The cells thus transfected were cultivated in the presence of various concentrations of hygromycin in order to select the cells replicating the transfected transcript.

The cell multiplication in the presence of different concentrations of hydromycin and the replicative activity of the mini-genomes were analysed simultaneously over one month. At regular intervals, Huh7/NS3-5B cells transfected or not by the 5UTR-H2AE-3UTR transcripts and in the presence of different concentrations of hygromycin where tripsinated and put back in suspension at a concentration 0.5×10⁶ to 1×10⁶ cells/ml in a 2 mM EDTA phosphate buffer. The cell suspensions obtained were then analysed by flow cytometry at 488 nm in order to detect the expression of EGFP and using an FACSCalibur® apparatus (BECKTON).

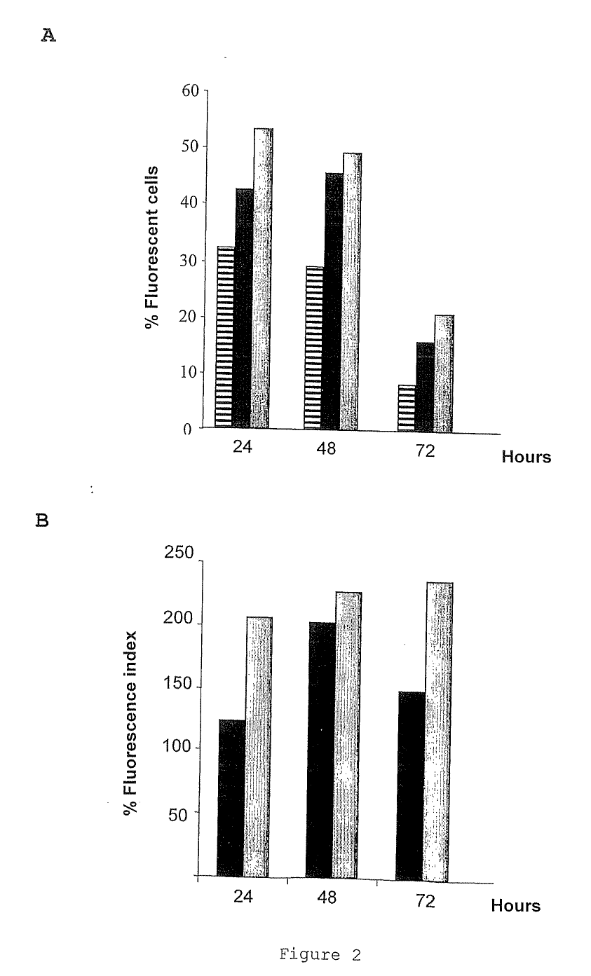

FIG. 2A shows the percentage of Huh (striped), Huh7/NS3-5B (black) and Huh7/Rep5.1 (grey, cf 3) fluorescent transfected cells 24, 48 and 72 hours after transfection. FIG. 2B shows the fluorescence index corresponding to the fluorescence ratio between the Huh7 and Huh7/NS3-5B (black) transfected cells and between the Huh 7 and Huh7/Rep5.1 (grey, cf 3) transfected cells. The results show that the percentage of cells showing a resistance to hygromycin is correlated with the percentage of cells expressing EGFP (FIGS. 2A and 2B), the said percentages increasing conjointly during the month of culture. In conclusion, the cells of the Huh7-NS3-5B cell line, which constituently express the NS3-NS5B polyprotein, allow production of an effective replication complex for replication of the mini-genome.

### 3) Replication Capacity of Negative-Polarity Minimal Genomic RNA Derived from HCV in an Infected Cell Line

The study model currently used for studying replication in infected cells is that of replicons. In the context of this analysis, we used cells from the Huh7 cell line expressing the replicon Rep5.1 of the HCV of genotype 1b (Huh7/Rep5.1; Kreiger et al, J Virol, vol 75, p: 4614-4624). Huh7/Rep5.1 and Huh7 cells were transfected by 1 μg of positive-polarity 5UTR-H2AE-3UTR transcripts according to the protocol described at 2-2. The expression of the EGFP protein was analysed after 24, 48 and 72 hours by flow cytometry (FIG. 2A). In order to take into account the variations in efficacy of translation, the expression of the EGFP protein is represented as the percentage expression of the EGFP by the Huh7/Rep5.1 cells with respect to the level of expression of the Huh7 cells.

The results show that the production of EGFP protein decreases after 48 hours of culture (FIG. 2A), which suggests degradation of the transcripts by the cells. However, this reduction is lower in the case of Huh7/Rep5.1 and Huh7/NS3-5B cells compared with the Huh7 cells, which suggests replication of the RNA by the replication complex of the HCV. The results obtained concerning the level of expression of the EGFP in the Huh7/Rep5.1 or Huh7/NS3-5B cells were normalised with respect to the level of expression of the EDFP obtained in the Huh7 cells (100%; FIG. 2B). The values higher than 100% indicate that the degradation observed in the Huh7 cells is compensated for by the replication of the minimal genomic RNA in the Huh7/Rep5.1 and Huh7/NS3-5B cells, suggesting that the replication complex issuing from the replicon is functioning effectively. The results also show that the level of expression of the EGFP increases during the first three days from 206 to 238% in the cells expressing the replicon, whereas in the Huh7/NS3-5B cells the level of expression increases for the first two days from 123 to 203%, and then decreases on the third day.

Since the Huh7/Rep5.1 cells are cultivated under selection pressure unlike the Huh7/NS3-5B cells, the difference in expression of EGFP between these two cell lines probably reflects a lesser cell permissiveness of the Huh7/NS3-5B cells in the prior absence of the selection pressure. In conclusion, the results show that the minimal genomic RNA replicates effectively in the two cell lines Huh7/Rep5.1 and Huh7/NS3-5B tested, which express a replication complex of the HCV of genotype 1b.

### 4) Evaluation of the Efficacy of a Suicide Vector Corresponding to a Minimal Genomic RNA

The above experiments show that the negative-polarity genomic RNAs derived from HCV were capable of replicating in cells hosting a replicon. In order to test the capacity of such a negative-polarity genomic RNA to destroy the cells infected by HCV, a chimeric genomic RNA was constructed as before in which the transgene corresponded not to the EGFP but to the gene coding for the A chain of the ricin framed by the 5′UTR and 3′UTR regions of the HCV (5UTR-Ric-3UTR). The gene of the A chain of the ricin was chosen as the suicide gene on the basis of its high toxicity vis-à-vis the eukaryotic ribosomes of the target cells infected by HCV (Eiklid et al, Exp Cell Res, vol 126(2), p: 321-6, 1980; Olsnes and Kozlov, Toxicon, vol 39(11), p: 1723-1728, 2001), and for its non-diffusiveness in the absence of the B chain, thus preserving the healthy surrounding cells that do not express the replication complex of HCV.

4.1: Construction of a Suicide Vector Corresponding to a Negative-Polarity Minimal Genomic RNA Derived from HCV:

The vector plasmid pGEM-T/5UTR-Ric-3UTR was constructed as follows:
The gene coding for the A chain of the ricin was obtained by PCR amplification using the primers Ric_Start (SEQ ID No 21: CACACAGGATCCATATTTCCCAAACAATACCCAATC) and Ric_Stop (SEQ ID No 22: ATATATTCTAGATTACTAAAACTGTGAGCTCGG).
The gene coding for chain A of the ricin was introduced into the plasmid pGEM-T/5UTR-EGFP-3UTR exchanging the gene of the EGFP for that of the A chain of the ricin and the sites BamHI and XbaI, producing the plasmid pGEM-T/5UTR-Ric-3UTR (FIG. 1C). The promoter of the phage T7 necessary for the transcription was introduced by PCR. In order to transcribe the (+) strand the primers T7-5UTR (SEQ ID No 17) and 3UTR-STOP (SEQ ID No 18) were used. In order to transcribe the (−) strand the primers T7-3UTR (SEQ ID No 19) and 5UTR-Start (SEQ ID No 20) were used.

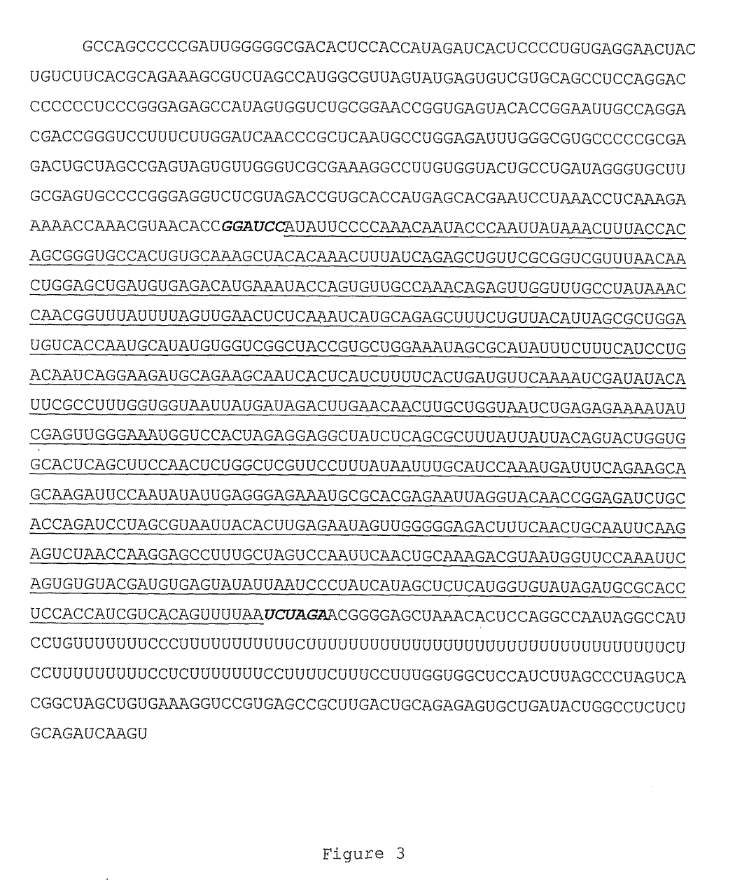

Finally, the negative-polarity minimal genomic RNA was obtained by transcription of 1 μg of PCR product using T7 polymerase (AMBION) following the instructions of the manufacturer. The negative-polarity minimal genomic RNA obtained is formed, starting from end 5′ to end 3′, by the complementary sequences of: the non-coding region 3′ of the HCV (3′UTR), the ricin A sequence, the first 27 nucleotides of the sequence coding for the capsid protein, and finally the non-coding region 5′ of the HCV. A positive-polarity minimal genomic RNA was also produced according to the same protocol and in vitro transcription with the T3 polymerase (AMBION) following the instructions of the manufacturer. FIG. 3 shows the sequence of the positive-polarity genomic RNA with the ricin A-chain sequence (underlined) framed by the cloning sites (bold and italics) and the 5′UTR (top) and 3′UTR (bottom) regions.

4-2: Replication of the Suicide Vector in the Huh7/Rep5.1 (Model for the Infected Cells) and Huh7/Ns3-58 Cell Lines:

Huh7/Rep5.1 and Huh7/NS3-5B cells were transfected by the negative-polarity minimal genomic RNA obtained at 4-1 according to the protocol described at 2-2. By way of control, Huh7 cells were transfected by the negative-polarity minimal genomic RNA obtained at 4-1 according to the same protocol. Also by way of control, the same transfection experiments were carried out with the positive-polarity minimal genomic RNA obtained at 4-1.

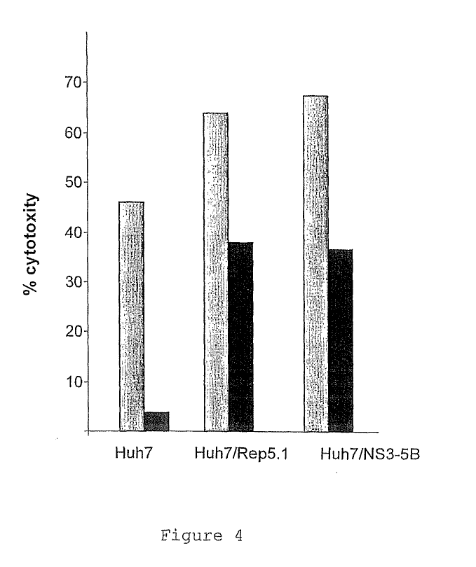

In the case where the said negative-polarity minimal genomic RNA will be replicated by the RNA-dependent RNA polymerase of the hepatitis C virus in a positive-polarity strand, the production of ricin (chain A) would then be obtained by virtue of the IRES sequence present within the 5′UTR region of the HCV and present in the positive-polarity strand. FIG. 4 shows the cytoxicity 48 hours after transfection of Huh7, Huh7/Rep5.1 or Huh7/NS3-5B cells by the negative-polarity (black) or positive-polarity (grey) minimal genomic RNA.

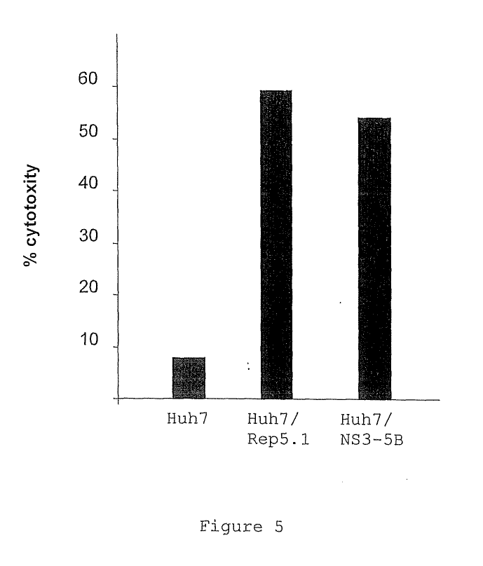

The results show that the transfection of the said negative-polarity minimal genomic RNA causes death of the Huh7/Rep5.1 Cells (38%) and Huh7/NS3-5B cells (36%), whereas the Huh7 cells are only slightly affected by the said negative-polarity minimal genomic RNA (4% are dead cells) (FIG. 4). FIG. 5 corresponds to the standardisation of the cytotoxicity percentage of the negative-polarity minimal genomic RNA taking as a transfection reference (100%) the cytotoxicity percentage of the positive-polarity minimal genomic RNA.

Standardisation of the results show that the negative-polarity minimal genomic RNA causes the death of 59% of the transfected Huh7/Rep5.1 cells and 54% of the transfected Huh7/NS3-5B cells, whereas only 9% of infected Huh7 cells are affected (FIG. 5). In conclusion, the results show that the suicide vector developed, corresponding to an RNA molecule derived from the HCV and of negative polarity, is effective for targeting and destroying the cells expressing the replication complexes of the HCV of genotype 1b. The inventors have also been able to show that the same suicide vector developed is effective for targeting and destroying (close on 90%) of the Huh7 cells infected by an HCV virus of the genotype 2a (JFH-1, Zhong et al, Proc Natl Acad Sci, USA, vol 102(26), p: 9294-9, 2005).

### 5) Evaluation of the Efficacy of a Vector Stimulating the Antiviral Cell Response Corresponding to a Minimal Genomic RNA

In order to test the capacity of such a negative-polarity genomic RNA to induce an antiviral response, a chimeric genomic RNA was constructed as described previously, in which the transgene corresponded to the gene coding either for alpha interferon (IFN-α), or for Regulatory Factor Interferon (IRF-1) by the 5′UTR and 3′UTR regions of the HCV (5UTR-Ric-3UTR).

5-1: Construction of a Vector Corresponding to a Negative-Polarity Minimal Genomic RNA Derived from the HCV:

The vector plasmids pGEM-T/5UTR-IFN-3UTR and pGEM-T/5UTR-IRF1-3UTR were constructed as follows:
The gene coding for IFN-α was obtained by PCR amplification using the primers IFN-Bam (SEQ ID No 23: ATATATGGATCCGCCCTGTCCTTTTCTTTACTGATGG) and IRF1-Xba (SEQ ID No 24: ATATATTCTAGATCAATCCTTCCTCCTTAATATTTTT TGC.
The gene coding for IRF-1 was obtained PCR amplification using the primers IRF1-Bam (SEQ ID No 25: ATATATGGATCCCCCATCACTCGGATGCGCATG) and IRF1-Xba (SEQ ID No 26: ATATATTCTAGACTACGGTGCACAGGGAATGGC).
The genes coding for IFN-α and IRF-1 were introduced into the plasmid pGEM-T/5UTR-EGFP-3UTR at the sites BamHI and XbaI, producing the plasmid pGEM-T/5UTR-IFNα-3UTR and pGEM-T/5UTR-IRF1-3UTR. The promoter of the T7 phage necessary for transcription was introduced by PCR. In order to transcribe the (+) strand the primers T7-5UTR (SEQ ID No 17) and 3UTR-STOP (SEQ ID No 18) were used. In order to transcribe the (−) strand the primers T7-3UTR 9SEQ ID No 19) and 5UTR-Start (SEQ ID No 20) were used.

Finally, the negative-polarity minimal genomic RNA was obtained by transcription of 1 μg of PCR product using T7 polymerase (AMBION) following the instructions of the manufacturer. The negative-polarity minimal genomic RNA obtained is formed, starting from the 5′ end towards the 3′ end, by the complementary sequences of: the non-coding 3′ region of the HCV (3′UTR), the sequence of IFN-α or IRF-1, the first 27 nucleotides of the sequence coding for the capsid protein, and finally the non-coding region 5′ of the HCV. A positive-polarity minimal genomic RNA was also produced in accordance with the same protocol and in vitro transcription with the T3 polymerase (AMBION) following the instructions of the manufacturer.

5-2: Replication of the Vector Stimulating the Antiviral Cell Response in the Huh7/Rep5.1 Cell Lines (Model for the Infected Cells):

Huh7/Rep5.1 cells were transfected by the negative-polarity minimal genomic RNA obtained at 5-1 according to the protocol described at 2-2. By way of control, Huh7/Rep5.1 cells were transfected by the positive-polarity minimal genomic RNA 5UTR-EGFP-3UTR obtained at 2-1, step 1, according to the same protocol. Also by way of control, the same transfection experiments were carried out with the positive-polarity minimal genomic RNA obtained at 5-1. In the case where the said negative-polarity minimum genomic RNA is replicated by the RNA-dependent RNA polymerase of the hepatitis C virus as a positive-polarity strand, the production of IFN-α or IRF-1 would then be obtained by virtue of the IRES sequence present within the 5′UTR region of the HCV and present in the positive-polarity strand.

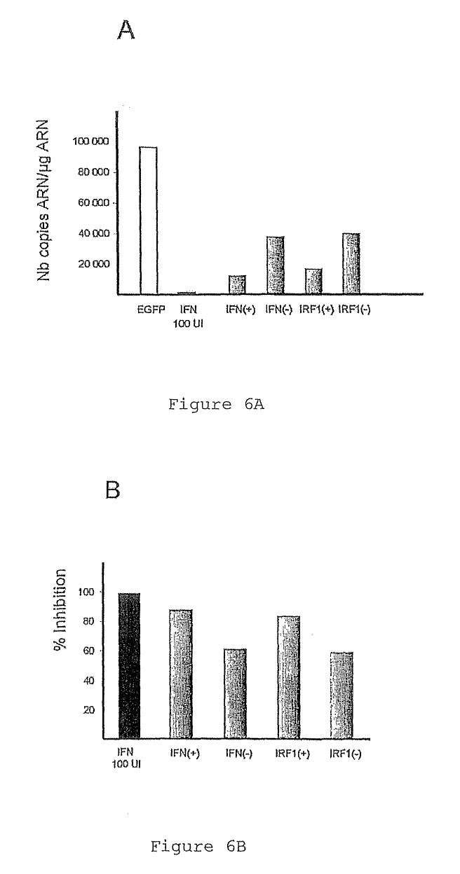

FIG. 6 shows the effects of the mini-genomes IFN and IRF on the Rep 5.1 replicon. The Huh7/Rep5.1 cells were transfected by the positive (+) or negative (−) polarity minimal genomic RNAs expressing IFN-α or IRF-1. FIG. 6A illustrates the number of RNA copies of the replicon measured by quantitative RT-PCR in real time reported per μg of total RNA after 48 hours of culture. The minimal genomic RNA 5UTR-ECFP-3UTR was transfected as a reference. One hundred UI of IFN-α was added to the cell culture (IFN 100 UI) as a positive reference. FIG. 6B illustrates the percentage inhibition of the replication of the Rep 5.1 replicon calculated by taking the minimal genomic RNA 5UTR-EGFP-3UTR as a replication reference.

The results show that the transfection of the said negative-polarity minimal genomic RNAs reduces the number of copies of genomic RNA of the replicon by 86% for the (+)RNA expressing IFN-α, 62% for the (−)RNA expressing IFN-α, 83% for the (+)RNA expressing IRF-1 and 60% for the (−)RNA expressing IRF-1 (FIG. 6B). In conclusion, the results show that the vectors stimulating the antiviral cell response, corresponding to a molecule of RNA derived from the HCV and of negative-polarity, are effective for targeting the cells expressing the replication complex of the HCV and reducing the number of genomic RNAs of the replicon.

1-30. (canceled)

31. A single-strand RNA molecule, which comprises, starting from the 3′ end towards the 5′ end:
(i) a complementary nucleic acid sequence of all or part of the 5′ non-coding region (5′UTR) of the RNA (+) strand of an RNA virus replicating by virtue of an RNA-dependent RNA polymerase, wherein said complementary nucleic acid sequence allows the replication of the said single-strand RNA molecule through the replication complex of said virus;
(ii) possibly the complementary nucleic acid sequence of a nucleic acid sequence corresponding to an internal entry site of the ribosome (IRES);
(iii) the complementary nucleic acid sequence of the nucleic acid sequence of a suicide gene or of a gene coding for a protein interfering with the replication of said virus; and
(iv) possibly a nucleic acid sequence complementary to the non-coding 3′ region (3′UTR) of the RNA of said virus.

32. A single-strand RNA molecule according to claim 31, wherein the said RNA virus replicating by virtue of an RNA dependent RNA polymerase is selected from the group comprising: the family of Flaviviridae, Cystoviridae, Birnaviridae, Reoviridea, Coronaviridae, Togaviridae, Arterivirus, Astroviridea, Caliciviridae, Picornaviridae, Potyviridae, Orthomyxoviridae, Filoviridae, Paramyxoviridae, Rhabdoviridae, Arenaviridea and Bunyaviridae.

33. A single-strand RNA molecule according to claim 31, wherein said RNA virus replicating by virtue of an RNA-dependent RNA polymerase is selected from the group comprising: the dengue virus, the yellow fever virus, the West Nile virus and the bovine diarrhoea virus.

34. A single-strand RNA molecule according to claim 31, further comprising starting from the 3′ end towards the 5′ end:
(i) a nucleic acid sequence complementary to all or part of the non-coding 5′ region (5′UTR) of the genomic RNA ((+) strand) of the hepatitis C virus (HCV), wherein said complementary nucleic acid sequence allows replication of said single-strand RNA molecule through the replication complex of the HCV;
(ii) the complementary nucleic acid sequence of a nucleic acid sequence corresponding to an internal entry site of the ribosome (IRES);
(iii) the complementary nucleic acid sequence of the nucleic acid sequence of a suicide gene or of a gene coding for a protein interfering with the replication of the HCV virus; and
(iv) possibly, a nucleic acid sequence complementary to the non-coding 3′ region (3′UTR) of the genomic RNA ((+) strand) of the HCV.

35. The single-strand RNA molecule according to claim 34, wherein said complementary nucleic acid sequence comprises at least the complementary sequence of a sequence comprising stem-loop domains I and II (5′UTR-dI and 5′UTR-dII) of the said 5′UTR region.

36. The single-strand RNA molecule according to claim 34, wherein said complementary nucleic acid sequence comprises at least the complementary sequence of a sequence comprising the stem-loop domains I, II and III (5′UTR-dI, 5′UTR-dII and 5′UTR-dIII) of the said 5′UTR region.

37. The single-strand RNA molecule according claim 34, wherein said complementary nucleic acid sequence comprises at least the complementary sequence of the sequence going from position 1 to 120 of the genomic RNA ((+) strand) of the hepatitis C virus.

38. The single-strand RNA molecule according to claim 34, wherein said complementary nucleic acid sequence comprises at least the complementary sequence of the sequence going from going from position 1 to 150 of the genomic RNA ((+) strand) of the hepatitis C virus.

39. A single-strand RNA molecule according to claim 34, wherein said 5′UTR region corresponds to the 5′UTR region of the genomic RNA of a hepatitis C virus of the genotype 1, 2, 3, 4, 5 or 6, or to a sequence derived therefrom, preferably to the 5′UTR region of the genomic RNA of a hepatitis C virus of genotype 1.

40. The single-strand RNA molecule according to claim 37, wherein said complementary nucleic acid sequence comprises at least the complementary sequence of the sequence going from position 1 to 120 of the sequence SEQ ID No: 1.

41. The single-strand RNA molecule according to claim 37, wherein said complementary nucleic acid sequence comprises at least the complementary sequence of the sequence going from position 1 to 150.

42. The single-strand RNA molecule according to claim 37, wherein said complementary nucleic acid sequence comprises at least the complementary sequence of the sequence going from position 1 to 341 of the sequence SEQ ID No: 1 or a sequence derived therefrom.

43. The single-strand RNA molecule according to claim 31, in which the nucleic acid sequence corresponding to an internal entry site of the ribosome (IRES) is of eukaryotic origin or viral origin.

44. The single-strand RNA molecule according to claim 43, wherein the nucleic acid sequence corresponding to an internal entry site of the ribosome (IRES) is of viral origin.

45. The single-strand RNA molecule according to claim 43, wherein the nucleic acid sequence corresponding to the IRES sequence of the hepatitis C virus, comprises the stem-loop domains II to IV (5′UTR-dII to 5′UTR-dIV) of the 5′UTR region of the genomic RNA ((+) strand) of the hepatitis C virus.

46. The single-strand RNA molecule according to claim 44, wherein the IRES sequence is the IRES sequence of the hepatitis C virus and in that it comprises the sequence going from position 30 to 355 of the genomic RNA ((+) strand) of the hepatitis C virus.

47. The single-strand RNA molecule according to claim 44, wherein the IRES sequence is the IRES sequence of the hepatitis C virus and in that it comprises the sequence going from position 25 to 385 of the genomic RNA ((+) strand) of the hepatitis C virus.

48. The single-strand RNA molecule according to claim 46, wherein said IRES sequence corresponds to the IRES sequence of the genomic RNA ((+) strand) of a hepatitis C virus of genotype 1, 2, 3, 4, 5 or 6, or a sequence derived therefrom, preferably to the IRES sequence of a hepatitis C virus of genotype 1.

49. The single-strand RNA molecule according to claim 46, wherein said nucleic acid sequence complementary to the said IRES region of the hepatitis C virus corresponds to the complementary sequence of the sequence going from position 30 to 355 of the sequence SEQ ID No: 1 or a sequence derived therefrom.

50. The single-strand RNA molecule according to claim 46, wherein said nucleic acid sequence complementary to the said IRES region of the hepatitis C virus corresponds to the complementary sequence of the sequence going from position 25 to 370 SEQ ID No: 1 or a sequence derived therefrom.

51. The single-strand RNA molecule according to claim 46, wherein said nucleic acid sequence complementary to the said IRES region of the hepatitis C virus corresponds to the complementary sequence of the sequence going from position 20 to 386 of the sequence SEQ ID No: 1 or a sequence derived therefrom.

52. The single-strand RNA molecule according to claim 34, wherein said single-strand RNA molecule comprises the complementary sequence of the sequence going from position 1 to 355 of the genomic RNA ((+) strand) of the hepatitis C virus.

53. The single-strand RNA molecule according to claim 34, wherein said single-strand RNA molecule comprises the complementary sequence of the sequence going from position 1 to 370 of the genomic RNA ((+) strand) of the hepatitis C virus.

54. The single-strand RNA molecule according to claim 52, wherein said sequence of the genomic RNA ((+) strand) of the hepatitis C virus corresponds to the sequence of a hepatitis C virus of genotype 1, 2, 3, 4, 5 or 6, or a sequence derived therefrom.

55. The single-strand RNA molecule according to claim 52, wherein said sequence of the genomic RNA ((+) strand) of the hepatitis C virus corresponds to the sequence of a hepatitis C virus of genotype 1.

56. The single-strand RNA molecule according to claim 52, wherein said sequence of the said single-strand RNA molecule comprises the complementary sequence of the sequence going from position 1 to 355 of the sequence SEQ ID No: 1.

57. The single-strand RNA molecule according to claim 52, wherein said sequence of the said single-strand RNA molecule comprises the complementary sequence of the sequence going from position 1 to 370 of the sequence SEQ ID No: 1.

58. The single-strand RNA molecule according to claim 52, wherein said sequence of the said single-strand RNA molecule comprises the complementary sequence of the sequence going from position 1 to 386 of the sequence SEQ ID No: 1.

59. The single-strand RNA molecule according to claim 31, wherein the suicide gene codes for a protein product that causes the death of the cell in the presence or absence of drugs.

60. The single-strand RNA molecule according to claim 59, wherein the suicide gene codes for a protein product that causes the death of the cell in the presence of drugs such as the genes of HSV thymadine kinase (HSV1tk) or cytosine deaminase (CD).

61. The single-strand RNA molecule according to claim 59, wherein the suicide gene codes for a protein toxin causing the death of the cell such as bacterial exotoxins, fungal toxins, toxins of eukaryotic origin, vegetable origin or viral origin of the proteins derived therefrom.

62. The single-strand RNA molecule according to claim 61, wherein the suicide gene codes for a protein toxin of the RIP (Ribosome-Inactivating Protein) family.

63. The single-strand RNA molecule according to claim 62, wherein the suicide gene codes for a protein toxin of the RIP family of type 2.

64. The single-strand RNA molecule according to claim 62, wherein the suicide gene codes for chain A of a protein toxin of the RIP family of type 2 such as ricin, abrin, modeccin, volkensin, viscumin and the Shiga toxin.

65. The single-strand RNA molecule according to claim 63, wherein the suicide gene corresponds to the sequence SEQ ID No: 2 or to a derived sequence.

66. The single-strand RNA molecule according to claim 31, wherein the gene coding for a protein interferes with the replication of the virus codes for an interferon-regulating factor.

67. An RNA molecule according to claim 31, wherein said nucleic acid sequence complementary to the said 3′UTR region comprises the complementary sequence of the sequence SEQ ID No: 3 or a sequence derived from this.

68. An RNA molecule according to claim 31, wherein said single-strand RNA molecule corresponds to the complementary sequence of the sequence SEQ ID No: 4 or a derived sequence.

69. A nucleic acid vector comprising a nucleic acid molecule according to claim 31.

70. A pharmaceutical composition comprising a nucleic acid molecule according to claim 31.

71. A method for preventing or treating an infection by an RNA virus replicating by virtue of an RNA-dependent RNA polymerase comprising the administration of a nucleic acid molecule according to claim 31.

72. The method for preventing or treating according claim 71, wherein said RNA virus is hepatitis C virus and the administration is done to a patient in need thereof.

73. The method according to claim 71, wherein the administration is done by topical or parenteral route.

74. The method according to claim 73, wherein the administration is done by intramuscular, intradermal, intrahepatic or subcutaneous injection.
